(19) United States
(12) Patent Application Publication
(10) Pub. No.: US 2012/0271220 A1
(43) Pub. Date: Oct. 25, 2012

(54) METHOD AND COMPOSITION FOR THE TREATMENT OR AMELIORATION OF FEMALE GYNECOLOGICAL INFLAMMATION

(76) Inventors: Marika Cherfas, Brooklyn, NY (US); Igor Zolotukhin, St. Petersburg (RU); Daniel Cherfas, Brooklyn, NY (US)

(21) Appl. No.: 13/090,149

(22) Filed: Apr. 19, 2011

(51) Int. Cl. A61M 37/00

(52) U.S. Cl. 604/20

(57) ABSTRACT

A method for treating inflammation of female genital area, the method including preparing a photo-active mineral composition of bischofite, aleurolite, magnesite and piezoquartz. The photo-active mineral composition is then inserted into a patient's rectum or vagina, where a piezo-electric current is generated and fed to the photo-active mineral composition, which generates infrared radiation in response to the fed piezo-electric current.

### BACKGROUND

The Application generally relates to the medical field of treatments for female gynecological inflammation.

Generally, the term “inflammation” in modern medicine is defined as a condition of bodily organs or tissues that is accompanied by enlarged blood vessels, increased permeability of vessel walls and microbial seeding in the inflamed area. On the other hand, a prolonged vessel spasm in a particular organ or tissue not receiving adequate nutrients results in atrophy. If such spasm extends to the entire net of veins and other blood vessels, hypostasis and subsequent cancer may result as a form emulating centrifugal energy of tissue strain from overload with the stagnant blood.

Existing treatment methods do not effectively treat vaginal inflammation or endometriosis. For example, vaginal suppositories act locally within vagina but untreated endometriosis continues to create the microbial seeding inside the vagina through endometrial serous/purulent discharge. Further, as known in the art, often the cause of female gynecological inflammation is the dysbacteriosis of the lower part of the colon. This dysbacteriosis is primarily related to microbial seeding of soft tissues which may not be affected by locally applied medications. Moreover, many pregnant women have undiagnosed sexually transmitted diseases which negatively affect conception and development of the fetus. The only known method of treating such diseases, in case of their transmission to the fetus, is infrared radiation which has a deep penetrating effect.

Ineffective treatment of various female pathologies often results in microbial seeding of the egg at the time of conception or of the fetus during the pregnancy. In fact, according to Russian physicians, 90% of Russian babies are born with some kind of pathology which remains untreated at least until the end of secondary school (96% of school-age children in Russia have some kind of somatic illness).

Research of the reproductive cells indicates that sometimes pathogenic micro-fauna participates in fetus development. This results in severe morning sickness during pregnancy, and sometimes, in inferior health condition of the newborn.

One known method of treating affected organs and tissues involves irradiation with visible and thermal rays. In this method, various electro-resistant mineral materials are created which, under the influence of electric current, create infrared radiation. Use of irradiation with visible and thermal rays inside a bodily cavity is also known. However, these known methods use the grid electric current, which is known to cause regenerative mutation of plants and fungi, which sometimes turn poisonous when grown near high voltage electric lines.

Accordingly, there is a need in the art for an effective and safe treatment method for the described inflammations.

### SUMMARY

It is a general object of the present invention to provide a method for treating inflammation of female genital area, the method including preparing a photo-active mineral composition of bischofite, aleurolite, magnesite and piezoquartz. The photo-active mineral composition is then inserted into a patient's rectum or vagina, where a piezo-electric current is generated and fed to the photo-active mineral composition, which generates infrared radiation in response to the fed piezo-electric current.

The above aspects, advantages and features are of representative embodiments only. It should be understood that they are not to be considered limitations on the invention as defined by the claims. Additional features and advantages of the invention will become apparent in the following description and from the claims.

### DETAILED DESCRIPTION

In the preferred embodiment, materials capable of generating thermal or visible light radiation are used inside a woman's vagina or rectum. This radiation is created when the materials are fed electric current generated by collisions of piezo-active quartz forms.

Specifically, in a laboratory experiment, the following combination of materials was used: bischofite (MgCl₂.6(H₂O)), aleurolite (shungite), magnesite (MgCO₃) and piezoquartz, hereinafter collectively referred to as the BAMP mineral composition. When the bischofite, aleurolith, and magnesite powders are mixed, a photo-active substance is formed. Further, when mono-crystal quartz plates framed by a metallic mineral collide, electric current is generated. When this generated electric current is fed to the BAMP composition, the desired radiation is created.

All of the enumerated minerals, when used in water-preparation, remove spasms of constrictive and dilative type in blood vessels and in neuro-muscle tissues that generate the above mentioned signs of inflammation. Additionally, it was determined during the laboratory experiment that the radiation generated by the BAMP composition removes spasms resulting in inflammation, ulcer-generating spasms, and spasms in other vital organs which often result in strokes or heart infractions.

During the experiment, the BAMP composition formed as a plate was securely attached to a male penis and inserted into a female vagina. Further, during a separate experiment, BAMP composition was formed into an anal suppository (which is warmed up internally by piezoquartz discharges) and inserted into a patient's rectum. When these treatments were performed systematically for 2-3 months, they resulted in disappearance of symptoms of vaginitis, endometriosis and colorectal disbacteriosis.

Relative comparison of the effectiveness of the described anti-inflammatory influence suggests that when photo-active minerals and a source of piezo-electricity are framed with metallic minerals the term of treatment is further reduced. The metallic minerals are preferably formed by electrolysis from a solution of mineral acids. Further, because, during pregnancy, a fetus is subjected to the infrared radiation of mineral nature (as a result of the above mentioned insertion of BAMP composition during the mother's intercourse), reduction of somatic diseases of the newborn is expected.

It is also important to note that, during the BAMP irradiation, detoxification of the genital cells happens in fractions of a second. Such irradiation can potentially influence and determine the quality of the conception and subsequent development of the fetus under continuous regular support by mineral solar radiation.

While there have been shown and described and pointed out fundamental novel features of the invention as applied to preferred embodiments thereof, it will be understood that various omissions and substitutions and changes in the form and details of the devices and methods illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

What is claimed is:

1. A method for treating inflammation of female genital area, comprising the steps of:
preparing a photo-active mineral composition comprising bischofite, aleurolite, magnesite and piezoquartz;
inserting the photo-active mineral composition into a cavity selected from the group consisting of a rectum and a vagina; and
generating a piezo-electric current and feeding the piezo-electric current to the photo-active mineral composition, which generates infrared radiation in response to the fed piezo-electric current.

2. The method of claim 1, wherein a source of the piezo-electric current is framed by a metallic mineral.

3. The method of claim 2, wherein the metallic mineral is formed by electrolysis from a solution of a mineral acid.

4. The method of claim 1, wherein the photo-active mineral composition is framed by a metallic mineral.

5. The method of claim 4, wherein the metallic mineral is formed by electrolysis from a solution of a mineral acid.

6. The method of claim 1, wherein the piezo-electric current is generated by collision of quartz plates.
